(19) United States
(12) Patent Application Publication
(10) Pub. No.: US 2009/0060992 A1
(43) Pub. Date: Mar. 5, 2009

(54) Preparation of magneto-vesicles with DOPE/DDAB layers

(75) Inventors: Weili Luo, Orlando, FL (US); Kezheng Chen, Orlando, FL (US)

Correspondence Address: LAW OFFICES OF BRIAN S STEINBERGER, 101 BREVARD AVENUE, COCOA, FL, 32922, US

(73) Assignee: UNIVERSITY OF CENTRAL FLORIDA RESEARCH FOUNDATION, INC.,

(21) Appl. No.: 12/290,102

(22) Filed: Oct. 27, 2008

Related U.S. Application Data
(62) Division of application No. 10/431,683, filed on May 8, 2003.
(60) Provisional application No. 60/378,747, filed on May 8, 2002.

(51) Int. Cl. A61K 9/127; A61K 47/12; A61P 43/00

(52) U.S. Cl. 424/450; 514/784

(57) ABSTRACT

Magneto-vesicles with two different surfactants, i.e., Dioleoyl phosphatidylethanolamine (DOPE) and dimethyl dioctadecylammonium bromide (DDAB), were synthesized using size controllable magnetite nanoparticles (Dₘ=9 nm) as cores. From AFM measurements, the average sizes of vesicles and magneto-vesicles are approximately 316 nm and approximately 311 nm, respectively. These biocompatible magneto-vesicles have very good dispersity in aqueous solution and affinity to cells, rendering them potentially useful as magnetic carriers for field-guided drug delivery. Light-emitting dye molecules together with magnetic particles were encapsulated inside these vesicles. An experiment showed that disruption of the vesicles releases the encapsulated dye molecules, thus the principle of using the drug-carrying magneto-vesicles as a drug delivery agent that can be guided by applied magnetic field has been demonstrated.

This invention relates to magneto-vesicles, in particular to methods of preparing biocompatible magneto-vesicles with Dioleoyl phosphatidylethanolamine (DOPE)/Dimethyl dioctadecylammonium bromide (DDAB) layers, that have good dispersiblity in aqueous solutions, and are useful in drug delivery and hyperthermia as magnetic carriers, was funded in part under a U.S. NSF NIRT grant, and this invention claims the benefit of priority based on U.S. Provisional Application No. 60/378,747 filed May 8, 2002.

### BACKGROUND AND PRIOR ART

Magnetoliposomes/vesicles have been proposed in past years. See for example, C. Sangregorio, J. K. Wiemann, C. J. O'Connor, et al (1999) J Appl Phys 85(8): 5699-5701; A. A. Kuznetsov, V. I. Filippov, R. N. Alyautdin, et al (2001) J Magn Magn Mater 225:95-100; and M. Shinkai, M. Yanase, M. Suzuki, et al (1999) J Magn Magn Mater 194:176-184. Vesicles have been known to include applications where a drug, and the like, can be encapsulated inside. The subsequent interaction of the encapsulated magnetic core with a magnetic field has been known to be able to help with the drug delivery.

The following U.S. patents are related to the field of the invention disclosed hereafter:

U.S. Pat. No. 6,470,220 (Kraus, Jr. et al) discloses binding a cancer binding agent to magnetic nanoparticles and subsequent liposome encapsulation;
U.S. Pat. No. 6,468,505 (Lang, et al) shows that liposome can be formed of nanoparticles and phosphatidylethanolamine;
U.S. Pat. No. 6,461,586 (Eguchi, et al) shows a superparamagnetic iron oxide mixture dissolved in a sonicating chamber to produce magnetite;
U.S. Pat. No. 6,315,981 (Unger) discloses stabilizing compounds for nanospheres;
U.S. Pat. No. 6,251,365 (Bauerlein, et al) discloses magnetsomes with magnetic particles of 43-45 nm;
U.S. Pat. No. 6,217,849 (Tournier, et al) discloses phospholipid liposomes with diameter of 0.2 to 10 micrometers;
U.S. Pat. No. 6,133,047 (Elaissari et al) discloses superparamagnetic particles containing magnetic nanoparticles as fillers;
U.S. Pat. No. 5,545,395 (Tournier, et al) discloses a structure having an iron oxide core and outer layer of an ampiphatic compound and a non-ionic surfactant;
U.S. Pat. No. 5,389,377 (Chagnon, et al) discloses iron oxide coated with phospholipids; and,
U.S. Pat. No. 4,728,575 (Gamble, et al) is directed to the preparation of micellar particulate vesicles having paramagnetic material enclosed within the vesicles.

However, the problems with these prior approaches include one or more of the properties that the coatings are not easily dispersible in aqueous solutions, are not bio-suitable or cannot be disrupted close to the desired location in the human body where the activity of the enclosed drug is useful, and require huge magnetic field for drug delivery. Thus, there exists the need for solutions to the above problems with the prior art.

### SUMMARY OF THE INVENTION

The first objective of the invention is to provide magneto-vesicles with a coating that has very good dispersibility in aqueous solutions and affinity to the diseased cells into which the contained ingredient of the vesicle has useful activity.

The second objective of the invention is to provide magneto-vesicles with a layer which enables the vesicles to be useful in drug delivery and hyperthermia as magnetic carriers.

The third objective of the invention is to provide magneto-vesicles that are biocompatible and releaseable under the influence of an external field.

Preferred embodiments of the invention include a biocompatible magneto-vesicle, comprising: a core having a substance for being selectively released and nanosized magnetic materials; and, a biocompatible outer bilayer of Dioleoyl phosphatidylethanolamine (DOPE) and dimethyl dioctadecylammonium bromide (DDAB) about the core, whereby the biocompatible outer layer has dispersiblity in aqueous solutions and the method of preparing a biocompatible magneto-vesicles, comprising the steps of: mixing Dioleoyl phosphatidylethanolamine (DOPE) and dimethyl dioctadecylammonium bromide (DDAB) together; and, applying the mixture to nanosized magnetic particles for forming the magneto-vesicles whereby the magneto-vesicles are biocompatible and, if desired, including the step of imposing magnetite nanoparticles inside the outer covering layer.

Further objects and advantages of this invention will be apparent from the following detailed description of various embodiments which are illustrated schematically in the accompanying drawings.

### BRIEF DESCRIPTION OF THE FIGURES

FIG. 1 shows a novel procedure for controlling the size of magnetic nanoparticles enabling stability of colloids consisting of these particles.

FIG. 2 is a flow chart for the process of preparing a stable magnetic colloid with controllable particle size.

FIG. 3 is a flow chart for the novel process of preparing the vesicles of the invention.

FIG. 4 shows a Transmission Electron Microscope (TEM) image of magneto-vesicles.

FIG. 5a illustrates the cross section of a magneto-vesicle.

FIG. 5b shows how the cross section of the magneto-vesicle is disrupted by a surfactant.

FIG. 6 shows the change of fluorescence intensity of ruptured magneto-vesicles in the presence of a surfactant.

FIG. 7 is a schematic illustrating the drug delivery mechanism by magneto-vesicles.

### DESCRIPTION OF THE PREFERRED EMBODIMENTS

Before explaining the disclosed embodiments of the present invention in detail it is to be understood that the invention is not limited in its application to the details of the particular arrangements shown since the invention is capable of other embodiments. Also, the terminology used herein is for the purpose of description and not of limitation.

A. Process of preparing controllable magnetic nanostructures.

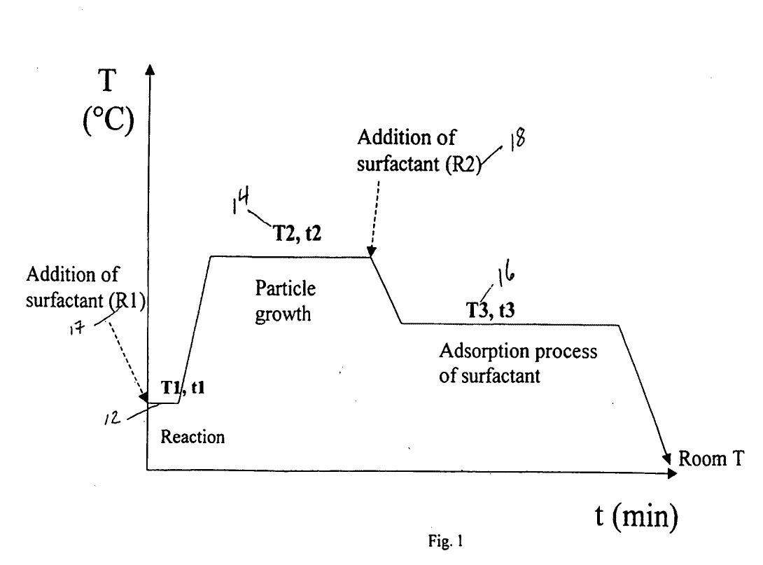

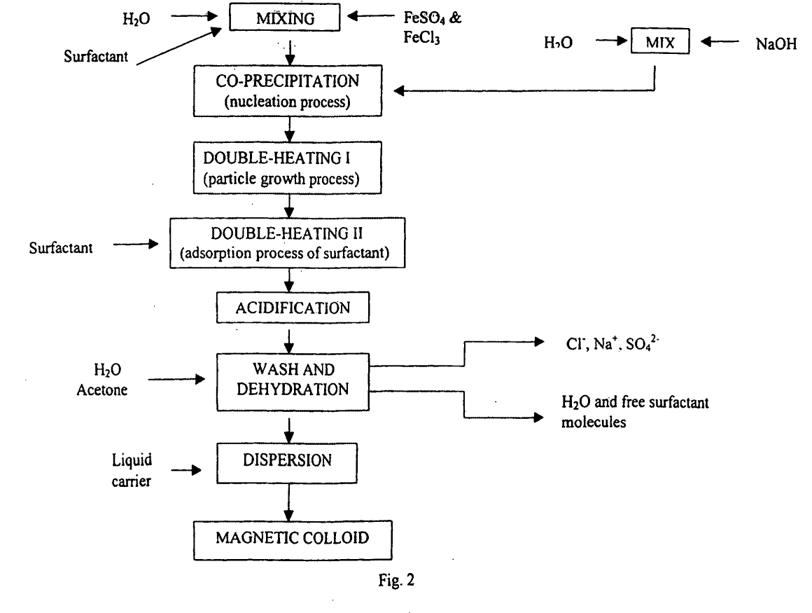

FIG. 1 illustrates the process of preparing controllable magnetic nanostructures for substance release from the magneto-vesicles which are later described hereafter. The preparation of the magnetic nanoparticles and subsequent magnetic colloids consisting of these particles utilizes a novel double-heating co-precipitation method which is illustrated in FIG. 1, where T₁ 12 is the temperature of the chemical reaction in this method, T₂ 14 is the heating temperature, T₃ 16 is the adsorption temperature where the surfactant molecules are adsorbed on particle surface, t₁, t₂, and t₃ are the times spent at above temperatures, respectively, R₁ 17 and R₂ 18 are the ratios of surfactants added at T₁ 12 and T₂ 14 accordingly (see both Example 1 following and the FIG. 2 flow chart). It has been discovered that the nanoparticle size can be controlled by varying the above-mentioned parameters.

During experimentation, the resulting suspension containing surfactant-coated magnetic nanoparticles were cooled to room temperature, then the pH value was adjusted. After decantation, the particle precipitate was washed several times with deionized water to remove other ions then again was washed with acetone. The washed precipitate was centrifuged to remove excess surfactants and residual water. After dried in vacuum overnight, a black precipitate (surfactant-coated magnetic particles) was obtained and was dispersed in a suitable solvents such as but not limited to: water and phosphate buffered saline in a sonication bath (Fisher FS-20) for a suitable time at room temperature.

B. Synthesizing controllable magneto-vesicles

Two types of lipid molecules were mixed during experimentation with nitrogen gas and then dried in vacuum to remove the residual solvent molecules. The controllable magnetic nanoparticles obtained in procedure A, discussed above and other useful organic or inorganic substances were added to the lipid molecules such as but not limited to: proteins, water-soluble medicine and light emitting dye molecules.

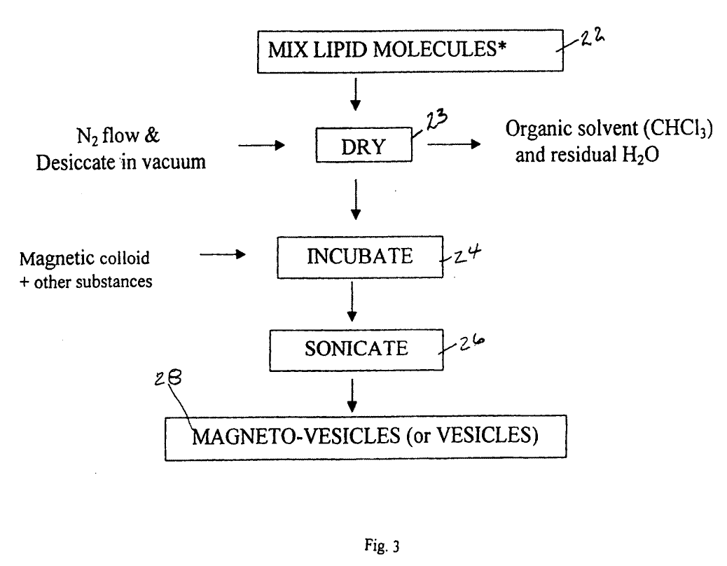

The combined system was incubated for several hours then sonicated for approximately an hour. The result is magneto-vesicles (MV) consisting of multiple magnetic nanoparticles together with other substances such as but not limited to: proteins, water-soluble medicine, and light-emitting dye molecules encapsulated in the MV. The free magnetic and other substances were separated from magneto-vesicles through the gel filtration method. The size of the MV can be further controlled by controlling the nanoparticle size, the nanoparticle concentration, the sonication temperature and sonication time. The sizes of MV's range from approximately 100 nm and up to approximately 1 micron. These procedure steps for control of the MV size is illustrated in FIG. 3.

### Example 1

Approximately 2 g of FeCl₃ 6H₂O and approximately 1.03 g of FeSO₄ 7H₂O were dissolved under N₂ in approximately 100 ml of 1M HCl solution with stirring, such that the molar ratio of Fe³⁺ to Fe²⁺ is 2. As the solution was heated to approximately 80° C., a solution of certain amount of surfactant (oleic acid, [OA]) in approximately 5 ml acetone was added (the amount added is defined as the surfactant ratio R.

$$R = \frac{[ O A ]}{[ {Fe}^{3 +} ] + [ {Fe}^{2 +} ]} ,$$

where [OA] represents the concentration of oleic acid). After adding approximately 8M NaOH solution to adjust the pH to approximately 11.5, magnetite particles were formed immediately.

For the magnetite nanoparticles, the optimal result is achieved (refer again to FIG. 1) with R₁=approximately 0.1, R₂=approximately 1, T₁=approximately 80° C., T₂=approximately 100° C., T₃=approximately 90° C., t₁=approximately 5 min, t₃=approximately 20 min, while t₂ varies from 5 minutes to one hour.

The resulting suspension containing surfactant-coated magnetic nanoparticles was cooled to room temperature, then approximately 1 mol HCl solution was added and the pH value was adjusted to approximately 2. After decantation, the particle precipitate was washed several times with deionized water to remove other ions (Cl⁻, SO₄²⁻, Na⁺), then again was washed with acetone. The washed precipitate was centrifuged to remove excess surfactants and residual water. After dried in vacuum overnight, a black precipitate (surfactant-coated magnetic particles) was obtained and was dispersed in a suitable solvent such as but not limited to water and phosphate buffered saline in a sonication bath (Fisher FS-20) for approximately 30 minutes at room temperature.

### Example 2

### Preparation of Magneto-Vesicles

FIG. 3 illustrates the step wise production of the magneto-vesicles of the invention. The first step is to mix lipid molecules 22 which in this example is Dioleoyl phosphatidylethanolamine (DOPE) and dimethyl dioctadecylammonium bromide (DDAB), purchased from Avanti Lipid. The lipid molecules also include other cationic lipids such as dioleoyldimethylammonium (DDAC), 1,2-dioleoyl-3-trimethylammonium propane (DOTAP) and N-{2,3-(dioleoyloxy)propyl]-N,N,N-trymethylammonium chloride (DOTMA) and phosphatidylcholines such as dioleoylphosphatidylcholine (DOPC), dilauroylphosphatidylcholine (DLPC), dimyristoylphosphatidylcholine (DMPC), dipalmitoylphosphatidylcholine (DPPC) and distearoylphosphatidylcholine (DSPC).

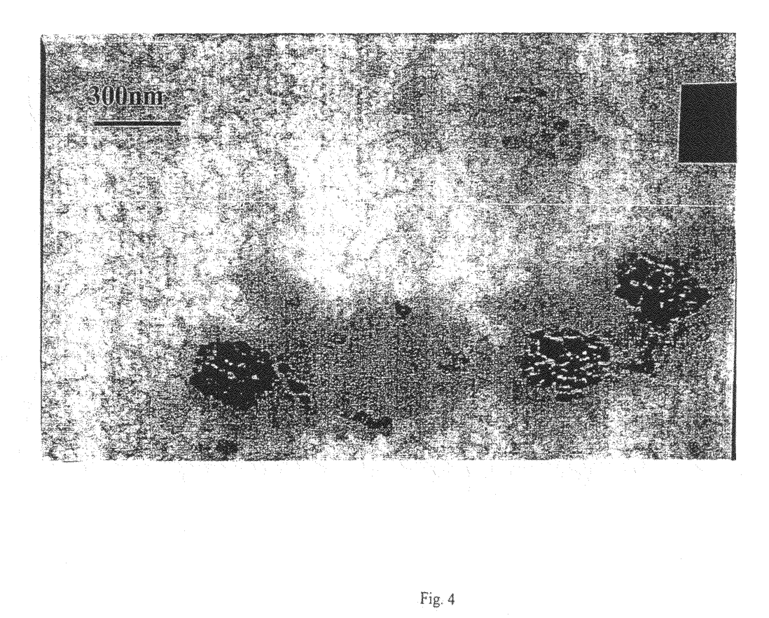

The next step is to dry 23 the mixture first by nitrogen flow then under vacuum, followed by incubation 24 with the magnetic nanoparticles of the 1^{st} Example and other useful substances such as but not limited to: proteins, water-soluble medicine and light-emitting dye molecules to be included in the magneto-vesicle followed by sonication 26. The sonication method was continuous at room temperature for approximately an hour with an energy input of approximately 40 KW. The resulting morphologies and size distributions of DOPE/DDAB vesicles and magneto-vesicles 28 were characterized by an Atomic Force Microscopy (AFM) examination as well as by Transmission Electron Microscopy (TEM) as shown in FIG. 4. Several dark spots within each MV of FIG. 4 show clearly encapsulation of multiple magnetic particles inside.

Referring again to the preparation of magneto-vesicles according to the invention, a series of DOPE/DDAB in chloroform stock solution were mixed at different concentration ratios such as but not limited to 1:1, 1:3, 1:5 and 1:7. The mixture of phospholipids was dried under nitrogen in a flask and was desiccated in vacuum overnight to remove the residual solvent in the dried film. A certain amount (such as but not limited to: 1 ml) of magnetic fluid containing magnetite particles having a size range of approximately 6 to approximately 10 nm was added to the dried film and the sample was incubated at approximately 40 degrees Centigrade for approximately 2 hours. The vesicle suspension was sonicated for approximately one hour in a sonicator. The resulting solution of magnetic vesicles was stored at approximately 4° C.

The vesicles with different concentration ratios of DOPE and DDAB were prepared at different temperatures such as but not limited to 22° C. and pH values such as but not limited to 7.

It was found that the aqueous vesicles suspension with DOPE to DDAB ratio of 1:1 is very stable to at room temperature up to approximately 3 months. With DOPE/DDAB (1:1, wt. %) as the surfactant, magneto-vesicles were synthesized using magnetite nanoparticles of Dₘ=approximately 9 nm, as the cores.

From AFM measurements, the size distributions of vesicles and magneto-vesicles can be described by the log-normal function. See R. A. Buhrman, C. G. Granqvist (1976) J Appl Phys 47:2200-2219. The average sizes of vesicles and magneto-vesicles are approximately 316 nm and approximately 311 nm, respectively.

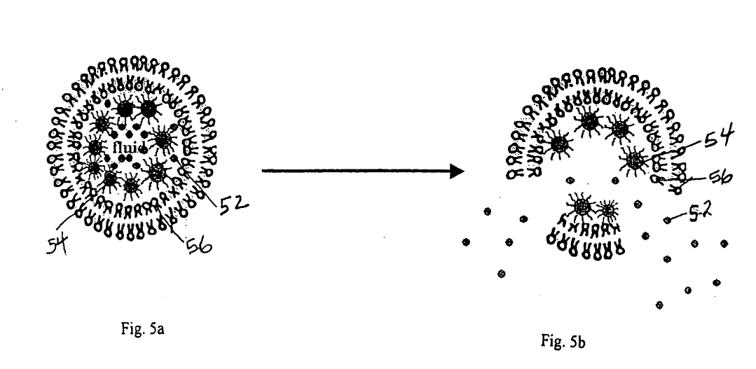

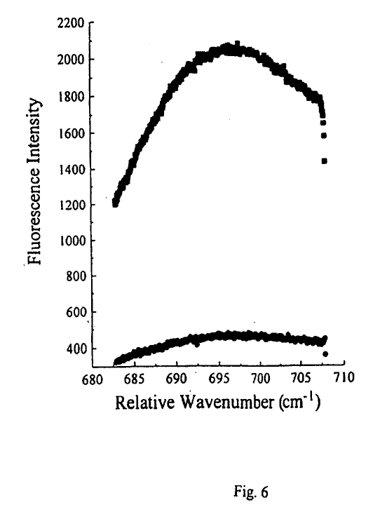

FIGS. 5a and 5b illustrates how a magneto-vesicle with the bi-layer of DOPE/DDAB 56 (which bi-layer consists of two layers, each layer having mixtures of DOPE/DDAB) containing encapsulated fluorescent dye molecules 52, ferrite nanoparticles 54 of the invention which when exposed to an approximately 10% aqueous solution of approximately 20 μl Triton X-100 disrupts the bilayer 56 coating allowing the dye 42 to escape and confirm the rupture of the magneto-vesicles as illustrated in FIG. 6. It is believed that the surfactant molecules and the lipid molecules of the magneto-vesicles tend to form micelles and thus destroying the vesicle bilayer.

FIG. 6 shows how the fluorescent intensity increases as the magneto-vesicles are ruptured. The lower curve represents the Fluorescence Intensity (FI) from the dye encapsulated inside the MV while the upper curve is the FI measured after the Triton X-100 was added, which leads to the disruption of the MV.

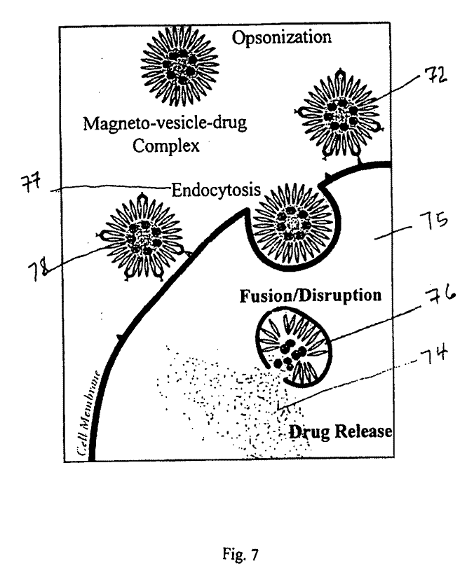

FIG. 7 illustrates one example showing the application for MV 72 as the mechanism for the drug delivery agent and drug release 74 inside a cell 75. The MV 72 can be guided to the target by a DC magnetic field after which the medicine inside the MV 76 can be released through Endocytosis 77 and Fusion processes. This local delivering method avoids damaging healthy cells. Encapsulating multiple magnetic nanoparticles 78 has the advantage of higher magnetic moment thus a smaller guiding magnetic field is required. It appears that the MVs function much as opsonins since they freely circulate in the blood. Thus the principle of using the drug-carrying magneto-vesicles 72 as a drug delivery agent that can be guided by applied magnetic field has been demonstrated.

The invention provides biocompatible magneto-vesicles that have good dispersiblity in aqueous solutions, and are useful in drug delivery and hyperthermia as magnetic carriers. There are numerous applications for use of the biocompatible magneto-vesicles of the invention including the encapsulation within or attachment to the biocompatible magneto-vesicles of substances such as medicine and therapeutic agents, antibodies, fusogenic peptides and other substances that can induce endocytosis and fusion.

The invention can also be useful for delivery of nutrition supplements; cosmetics and heating elements.

The vesicles of the invention makes possible its guidance to the desired location such as a tumor cell by application of an external field and/or rupture of the vesicles and release of the contents by contact with a cell membrane with certain pH value or by magnetic fusion (MF) or by an external field.

The magneto-vesicles of the invention makes possible controllable Magneto-Endocytosis (ME) and Magneto-Fusion (MF).

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

We claim:

1-14. (canceled)

15. A process of preparing biocompatible, controllable magnetic nanostructures, that form magneto-vesicles, with a core of multiple nanosized magnetic particles and a biocompatible outer bilayer about the core, using a first procedure (process A) for preparing nanosized magnetic particles of controlled size, and a second procedure (process B) for synthesizing the magnetic nanostructures, wherein process A uses co-precipitation and double-heating, comprising the steps of:
(a) dissolving ferric chloride (FeCl₃) and ferrous sulfate (FeSO₄) in hydrochloric acid (HCl) to provide a mixture (I);
(b) combining mixture (I) with a surfactant and heating to a temperature of approximately 80° C. for approximately 5 minutes to form mixture (II);
(c) adding sodium hydroxide (NaOH) to mixture (II) and heating mixture II to approximately 100° C. for a period of time from approximately 5 minutes to approximately one hour to allow for particle growth and form mixture (III);
(d) adding an amount of a surfactant to mixture III to form mixture IV;
(e) reducing the temperature of mixture IV to approximately 90° C. for approximately 20 minutes to allow adsorption of the surfactant and to form a suspension;
(f) allowing the suspension of step e) which contains surfactant-coated magnetic nanoparticles to cool to room temperature;
(g) adjusting the pH of the cooled suspension to approximately 2 to form a precipitate;
(h) washing and subsequently dehydrating the precipitate of step (g);
(i) centrifuging the dehydrated precipitate of step (h) to remove surfactant and residual water;
(j) drying the dehydrated precipitate of step (i) in a vacuum;
(k) dispersing the dried precipitate of step (j) in a solvent to form mixture (V) containing surfactant-coated magnetic nanoparticles for the preparation of magneto-vesicles using process B consisting of the steps of:
(l) mixing dioleoyl phosphatidylethanolamine (DOPE) and dimethyl dioctadecylammonium bromide (DDAB) together to provide a lipid mixture (VI);
(m) sonicating mixture (VI) together with magnetic particles in mixture (V) for approximately one hour at room temperature, whereby magneto-vesicles are provided which encapsulate multiple magnetic particles and are biocompatible.

16. The process according to claim 15 wherein the ferric chloride and ferrous sulfate are in a molar ratio of 2 to 1, respectively.

17. The process according to claim 15 wherein the precipitate in step g) is formed when a hydrochloric acid (HCl) solution is added and the pH value is adjusted to approximately 2.

18. The process according to claim 15 wherein the temperature in step c) is approximately 95 degrees centigrade and is held for from 2-30 minutes whereby iron particle growth is caused.

19. The process according to claim 15 wherein the reduced temperature in step e) is approximately 85 degrees centigrade and is held for adsorption of the surfactant.

20. The process according to claim 15 wherein the preparation of biocompatible magneto-vesicles includes the further steps of incubating useful substances selected from at least one of proteins, water-soluble medicine and light-emitting dye molecules with the dried magnetic nanoparticles from step j) forming a colloidal suspension of the controllable magnetic nanoparticles, followed by sonicating the colloidal suspension continuously at room temperature for approximately one hour to form a plurality of magneto-vesicles that encapsulate substances and are useful as a delivery agent that can be guided by applied magnetic field.
